(19) United States
(12) Patent Application Publication
(10) Pub. No.: US 2010/0145157 A1
(43) Pub. Date: Jun. 10, 2010

(54) COMPACT MONITOR AND A METHOD FOR MONITORING A USER

(76) Inventors: Reuven Nanikashvili, Ashdod (IL); Moshe Raines, Tel-Aviv (IL)

Correspondence Address: Pearl Cohen Zedek Latzer, LLP, 1500 Broadway, 12th Floor, New York, NY, 10036, US

(21) Appl. No.: 11/573,197

(22) PCT Filed: Jan. 22, 2007
(86) PCT No.: PCT/IL07/00079

§ 371 Date: Feb. 2, 2010

Related U.S. Application Data
(60) Provisional application No. 60/751,317, filed on Dec. 16, 2005.

(51) Int. Cl. A61B 5/00

(52) U.S. Cl. 600/300

(57) ABSTRACT

A compact monitor and a method for health monitoring. The compact monitor includes: at least one physiological sensor; a mass storage unit; and a connector, electrically coupled to the mass storage unit; wherein the connector is adapted to mechanically support the compact monitor when the connector is rigidly connected to a another device.

### RELATED APPLICATIONS

This patent application claims the priority of U.S. provisional patent Ser. No. 60/751,317 filing date Jan. 23, 2006.

### FIELD OF THE INVENTION

The invention relates to a compact monitor and method for monitoring a user.

### BACKGROUND OF THE INVENTION

Various health monitors were developed during the last decade. These health monitors can transmit information to a host computer, to a medical center and the like. A typical health monitor includes special hardware for gathering and processing physiological data. The following U.S patents and patent applications, which are incorporated herein by reference, provide a brief review of state of the art systems and devices: U.S patent application 2004/0027244 of Menard, titled “Personal medical device communication system and method”; U.S. Pat. No. 5,390,238 of Kirk, et al., titled “Health support system”; U.S. Pat. No. 5,566,676 of Rosenfeldt el al., titled “Pressure data acquisition device for a patient monitoring system”; U.S. Pat. No. 5,772,586 of Heinonen et al., titled “Method for monitoring the health of a patient”, U.S. Pat. No. 5,840,020 of Heinonen et al., titled “monitoring method and a monitoring equipment”; U.S. Pat. No. 5,983,193 of Heinonen et al., titled “patient's nursing apparatus and nursing system”; U.S. Pat. No. 6,366,871 titled “Personal ambulatory cellular health monitor for mobile patient” of Geva; U.S. Pat. No. 6,605,038 of Teller et al. and U.S. patent application serial number 2005/0245793 of Hilton et al.

USB flash drives are flash memory data storage devices integrated with a Universal Serial Bus (USB) interface. They are rigid, compact and, can store a large amount of information. USB flash drivers are commonly used for transferring data between computers. In many cases they are used to carry applications that run on a host computer connected to the USB flash drivers.

A typical USB flash driver includes a male USB connector and a housing in which a small printed circuit board, controller, flash memory integrated circuit and crystal oscillator are placed. The controller controls the access to the flash memory integrated circuit. The following U.S patents and patent applications, which are incorporated herein by reference, provide a brief review of state of the art USB flash drivers: U.S. patent application serial number 2006/0230226A1 of Meir et al., titled “Hard disk drive with optional cache memory”; U.S. patent application serial number 2006/0198202A1 of Erez, titled “Flash memory backup system and method”; U.S. patent application serial number 2006/0184724A1 of Meir el al., titled “NAND flash memory system architecture”; U.S. patent application serial number 2006/0107317A1 of Moran el al., titled “Selective protection of files on portable memory devices”; U.S. patent application serial number 2006/0004952A1 of Lasser titled “Method of managing a multi-bit-cell flash memory”; U.S. Pat. No. 6,986,030 of Shemueli et al., titled “Portable memory device includes software program for interacting with host computing device to provide a customized configuration for the program”; and U.S. patent application serial number 2005/0038983A1 of Moran titled “Novel flash memory arrangement”.

A context-based reminder is illustrated in U.S patent application serial number 2002/012035A1 of Tuomela et al. describes a context-based data logging and monitoring device that can provide context-based reminders.

Each of these mentioned above devices and methods provide partial solutions to dedicated problems.

There is a need to provide an efficient multi-purpose monitor and method for monitoring a user.

### SUMMARY OF THE INVENTION

A compact monitor that includes: at least one physiological sensor; a mass storage unit; and a connector, electrically connected to the mass storage unit; wherein the connector is adapted to mechanically support the compact monitor when the connector is rigidly connected to a another device.

A method for health monitoring, the method includes: storing a large amount of data at a mass storage unit that belongs to a compact monitor; gathering physiological information by at least one physiological sensor that belongs to the compact monitor; rigidly connecting a connector of the compact monitor to another device; wherein the connector provides mechanical support to the personal health monitor; wherein the connector is electrically coupled to the mass storage device.

A compact monitor that includes a processor connected to an alert circuit and at least one physiological sensor; wherein the health monitoring device is adapted to monitor a state of a user at a first monitoring level and evaluate whether a monitoring condition is fulfilled; and wherein in response to a completion of the monitoring condition the health monitoring device is adapted to generate a request, via the alert circuit, to the user, to change a monitoring level of the health monitoring device.

A method for health monitoring, the method includes: monitoring, by a compact monitor, a state of a user, at a first monitoring level, and evaluating whether a monitoring condition is fulfilled; and generating a request, via an alert circuit, to the user, to change a monitoring level of the health monitoring device, if the monitoring condition is fulfilled.

A compact monitor that includes: a processor connected to a mass storage unit and to a user interface; wherein the compact monitor is adapted to generate context-based reminders to carry out at least one predefined action, via the user interface, and wherein the compact monitor is adapted to determine a completion of at least one predefined action in response to an input provided to the compact monitor.

A method for monitoring a user, the method includes: generating, via a user interface of a compact monitor that comprises a processor coupled to a mass storage unit, context-based reminders to carry out at least one predefined action; and determining a completion of at least one predefined action in response to an input provided to the compact monitor.

### BRIEF DESCRIPTION OF THE DRAWINGS

Further features and advantages of the invention will be apparent from the description below. The invention is herein described, by way of example only, with reference to the accompanying drawings, wherein:

FIG. 1 illustrates a compact monitor, according to an embodiment of the invention;

FIG. 2 is a rear view of a compact monitor according to an embodiment of the invention;

FIGS. 3-4 illustrate the interior of the compact monitor of FIG. 1, according to an embodiment of the invention;

FIG. 5 is a block diagram of a compact monitor, according to an embodiment of the invention;

FIGS. 6a-6d illustrate multiple exemplary screens according to an embodiment of the invention; and

FIGS. 7-9 illustrate methods for monitoring a user, according to various embodiment of the invention.

### DETAILED DESCRIPTION OF THE DRAWINGS

According to an embodiment of the invention a compact monitor is provided. It is a consumer type product that can monitor a user as well as provide mass storage (such as USB flash drive) capabilities. The monitoring applications can utilize data, images, icons and even code stored at the mass storage unit.

The compact monitor can be connected to another device (such as a host computer) by a connector that is electrically connected to the mass storage unit and is adapted to mechanically support the compact monitor when the connector is rigidly connected to the receiving device. The other device can participate in the execution of various health monitoring applications, context based reminder and verification applications, can archive information stored at the compact monitor, download information to the compact monitor and the like. The compact monitor is connected to the other device in a rigid manner, by using the connector. Conveniently no wire or other flexible connecting component is required, and the size of the overall monitor is small, it can be less than few centimeters.

It is conveniently easier to carry the compact monitor, as it does not include any cable or other means that provide mechanical support to the compact monitor, other than the (compact) connector. Accordingly, the compact monitor can be carried by a person with minimal clothing.

According to an embodiment of the invention the compact monitor is adapted to monitor a state of a user at a first monitoring level and evaluate whether a specific, pre-defined monitoring condition is fulfilled. If the expected monitoring condition is fulfilled then the compact monitor is adapted to generate a request, via the alert circuit, to the user, to switch to another monitoring level of the health monitoring device that is suitable for monitoring the patient when said special expected monitoring condition is fulfilled.

Conveniently, the special, expected monitoring condition can represents a mental state of the user, an environmental condition, a heart beat of the user, a cardiac state of the user, a respiration of the user, a temperature of the user, and the like.

According to an embodiment of the invention the first monitoring level can be performed without contacting the user. The compact monitor can be placed in a pocket of the user's shirt, connected to the user's pants, placed within a purse or a handbag of the user and monitor the state of the user. The monitoring can include analyzing the voice of the user, evaluating the respiration of the user, evaluating whether the user runs, walks, sits or lies down, and the like. The change of the monitoring level from the first level to a second level of monitoring may require the user to perform a certain action such as to contact a sensor of the compact monitor and enable the compact monitor to perform one or more tests. Conveniently, the first level of monitoring is characterized by lower power consumption than the second level of monitoring, and the second level monitoring provides more accurate indications about the state of the user.

It is noted that the same compact monitor can be used to apply the first and second level monitoring although this is not necessarily so and each monitoring level can be applied by a different monitor.

The compact monitor can include one or more physiological sensors such as but not limited to heart beat rate sensor, temperature sensor, body fat analyzer, blood sugar sensor, and ECG sensor. The compact monitor can also include additional environmental sensors such as accelerometer, humidity sensor, location detector and the like. For simplicity of explanation the following figures illustrate a temperature sensor and two ECG electrodes.

The compact monitor can execute various health related applications as well as context based reminder and verifier applications. In some cases the context is also health related. Exemplary applications include: ECG analyzer, diet manager, fitness manager and drug reminder and verifier.

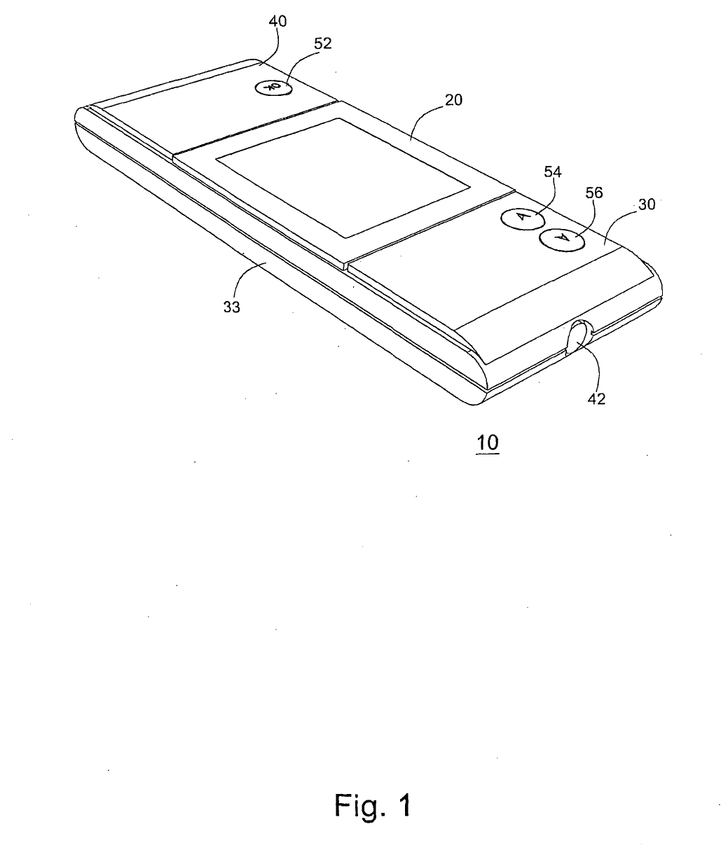

FIG. 1 is a three dimensional view of compact monitor 10 according to an embodiment of the invention.

Compact monitor 10 is relatively thin and long and has an oval shape. The inventors used a compact monitor that was 82 mm long, 8.5 mm wide and had a height of 25.6 mm. It is noted that the compact monitor can be of other dimensions without departing from the scope of the claims.

FIG. 1 illustrates housing 33 that has a front panel as well as multiple side panels. The front panel includes a display 20 positioned at the center of the front panel, control push buttons 52, 54 and 56, and ECG electrodes 40 and 30. ECG sensors 40 and 30 are positioned at both sides of display 20. First control push button 52 is positioned at the left side of display 20 while second and third push buttons 54 and 56 are positioned at the right side of display 20. Temperature sensor 42 is positioned at the right side of compact monitor 10.

Display 20 is conveniently positioned below a transparent cover.

Display 20 can be a black and white display, a color display, a graphical display and the like. The inventors used an OLED color display that has a color depth of 65,000 colors, resolution of at least 96×96 pixels, effective viewing area of about 20×20 mm and a power consumption of about 20 mA at battery voltage.

Second and third control push button 54 and 56 are used to browse between screens and menus displayed on display 20 while first control push button 52 is used for selecting items. It is noted that compact monitor 10 can include more than three control push buttons and that these control push buttons can be used for inputting various types of information. Compact monitor 10 can also include less than three control push button and can include input elements (such as knobs, a contact screen, vocal based control element, and the like) that differ from push buttons.

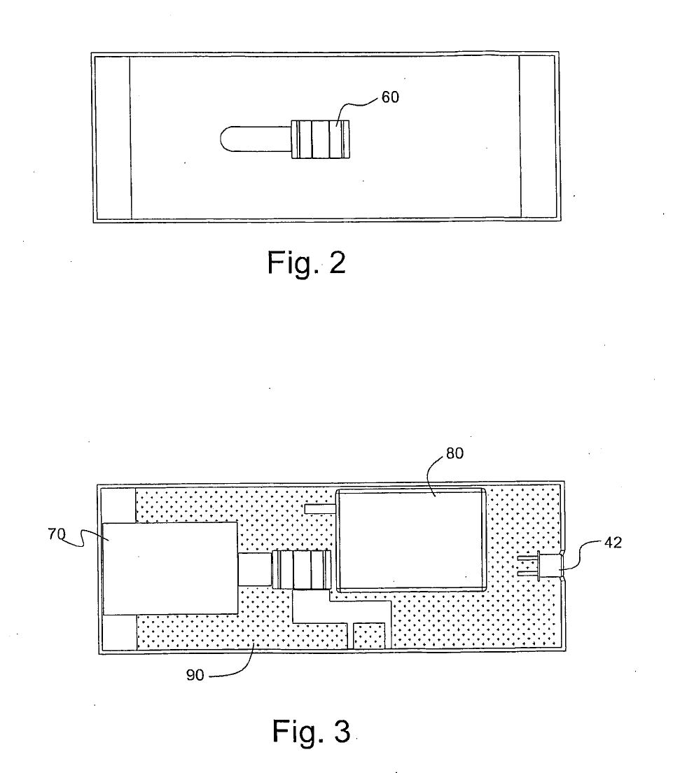

FIG. 2 is a rear view of compact monitor 10 according to an embodiment of the invention. The read panel of compact monitor 10 includes a control element such as slider 60 that is connected to a connector such as a movable male USB connector (such as male USB connector 70 of FIG. 3). Slider 60 is connected to the male USB connector and determines the position of the male USE connector. The Male USB connector 70 can slide through an aperture defined in the housing of compact monitor 10. Male USB connector 70 provides mechanical support to compact monitor 10 when the compact monitor is plugged to a host computer.

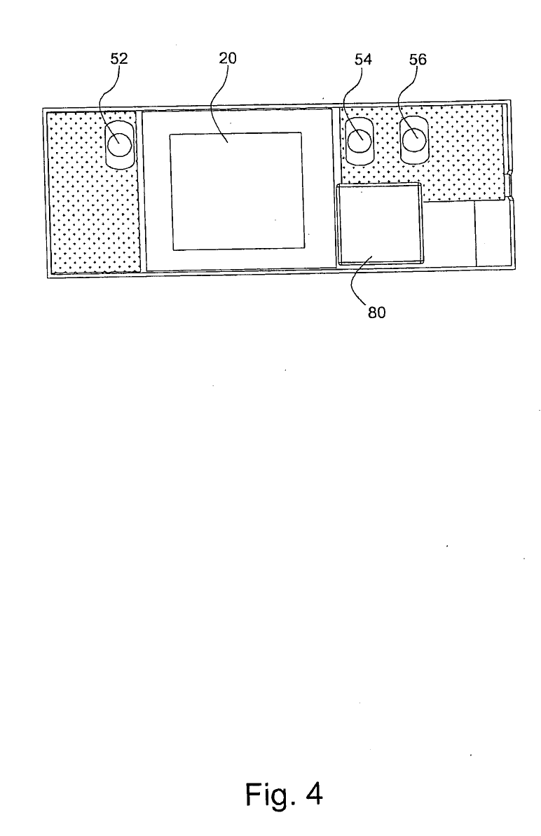

FIG. 3 and FIG. 4 illustrate the interior of compact monitor 10 according to an embodiment of the invention.

FIG. 4 provides a top view of the interior of compact monitor 10. Display 20 is positioned at the center of compact monitor 10. Battery 80 is positioned to the right of display 20. Second and third push buttons 54 and 56 are positioned above battery 80.

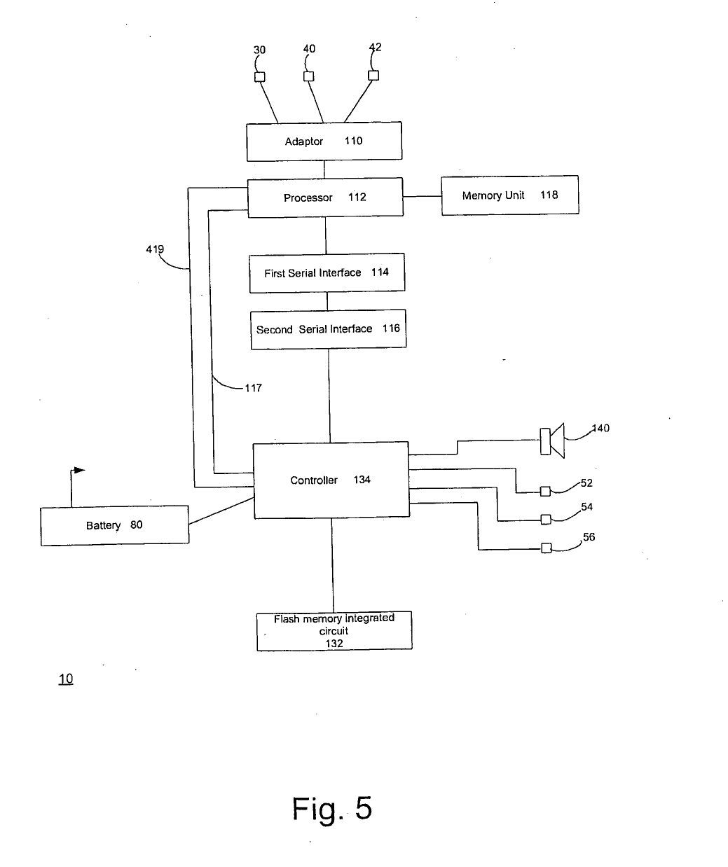

FIG. 3 provides a rear view of the interior of compact monitor 10. It illustrates temperature sensor 42, connector 70 (that can slide along an imaginary horizontal axis of compact monitor 10) and printed circuit board 90 that can be connected to display 20, temperature sensor 42, as well as to various integrated circuits and components, further illustrated in FIG. 5.

FIG. 5 is a block diagram of compact monitor 10 according to an embodiment of the invention.

According to an embodiment of the invention compact monitor 10 includes a single printed circuit board (PCB) 90 but according to another embodiment of the invention it includes a USB flash driver PCB and a health monitor PCB.

Compact monitor 10 includes multiple sensors 30, 40 and 42, adaptor 110, processor 112, small memory unit 118, controller 134, interrupt control lines 117 and 119, first serial interface 114, serial bus 115, second serial interface 116, non-volatile mass storage unit such as flash memory integrated circuit 132, buzzer 140, battery 80, display 20 and push control buttons 52-56.

Processor 112 is a high-level processor which is adapted to execute complex applications such as diet manager, drug reminder and the like. Sensors 30, 40 and 42 are connected to adaptor 110 that can convert analog detection signals provided by these sensors to digital detection signals that can be stored at memory unit 118 or at flash memory integrated circuit 132. These digital detection signals are processed by processor 112. Adaptor 110 includes at least one digital to analog converter and can process detection signals from one or more sensor concurrently. Additionally or alternatively, adaptor 110 can apply time division multiplexing techniques and process detection signals from a single sensor at a given point in time.

Adaptor 110 can include analog and/or digital circuitry that can amplify, filter and otherwise process the detection signals from sensors 30, 40 and 42. Adaptor 110 can include a medical sub-system such as the medical sub-system illustrated in U.S. Pat. No. 6,366,871 titled “Personal ambulatory cellular health monitor for mobile patient” of Geva, which incorporated herein by reference.

Processor 112 is connected to controller 134 via two interrupt control lines 117 and 119. Processor 112 is also connected to first serial interface 114 that is connected to serial bus 115. Second serial interface 116 is connected between serial bus 115 and controller 134. Serial interfaces 114 and 116 can be UART compliant but this is not necessarily so.

Controller 134 can perform at least one of the following functions: (i) controls the access to flash memory integrated circuit 132, (ii) controls various hardware components of compact monitor such as display 20, buzzer 140 and battery 20, (iii) receives inputs from control push buttons 52-56, (iv) control the charging of battery 20 and perform power management, (v) control the audio level of buzzer 140, the tone of buzzer 140 and also determine when to activate buzzer 140.

Compact monitor 10 can generate audio alerts, visual alerts or a combination of both. Accordingly, display 20 and/or buzzer 140 can be regarded as the alert circuit of compact monitor 10.

Interrupt control lines 117 and 119 can be used to send activations signals (exiting an idle or suspend mode) between controller 134 and processor 120.

Flash memory integrated circuit 132 stores multiple images and icons that can be displayed on display 20. It can also store context information that is used by a context based reminder and verifier application.

The inventors used a compact monitor 10 that utilized one hundred images, each including up to 96×96 color pixels, as well as twenty icons each including up to 10×12 color pixels. It is noted that these images and icons are utilized during the execution of various applications including health monitoring applications and context-based reminder and verifier applications. Some of the images can serve as background to text or other symbols.

Wealth related instructions, test results and reminders can be displayed on display 20 in various manners. For example, a user can be invoked to start a test by generating an audio alert (by buzzer 140) and/or by displaying a request to perform such a test. During the test display 20 can display and indication that indicates the current stage of the test (for example: “measurement being performed” . . . . “measurements signals are being processed” . . . “test results are being stored” . . . , and the like).

Display and/or buzzer 140 can be used to generate a request to the user to alter the monitoring level from a first monitoring level to a second monitoring level, and vice verse.

According to an embodiment of the invention compact monitor 10 is adapted to transmit physiological information gathered by one or more physiological sensor. The transmitted physiological information can be provided via USB connector 70 but this is not necessarily so. Compact monitor 10 can include a communication circuit that can include USB connector 70 but may, additionally or alternatively, include a wireless transmitter that can enable compact monitor 10 to transmit the physiological information. The communication circuit can utilize any communication protocol known in the art and is preferably a short range transmitter. It is noted that the communication circuit can also include a receiver for receiving information.

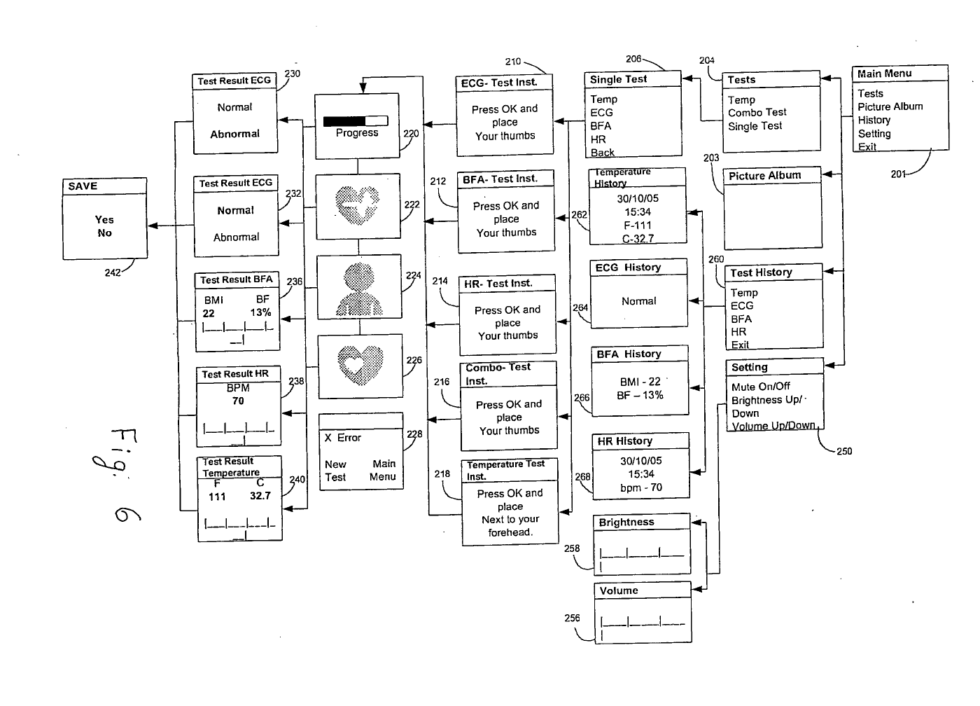

FIGS. 6a-6d illustrate multiple exemplary screens according to an embodiment of the invention.

First screen 201 displays the main menu. A user can select between performing a test (“tests”), viewing a picture album (“picture album”), reviewing results of old tests (“history”), define the settings of display 20 and buzzer 140 (“settings”) or exit (“exit”). If “tests” is selected then test screen 204 appears and the user can select between conducting a single test (“single test”), a combination of tests (“combo test”) or a temperature test (“temprature.”). If a single test is selected a single test menu screen 206 is displayed and the user can select between conducting a temperature test (“temperature.”), ECG test (“ECG”), BFA test (“BFA”), heart rate test (“HR”) or exit to the main menu (“menu”).

A selection of a test (single test or a combined test) will result in a display of a test instruction screen out of screens 210-218. Each of these test instruction screens includes the name of the selected test as well as instructions related to the test such as pressing the first push button 52 and then pressing ECG sensors 30 and 40.

During the test period a test progress image out of test progress images 220-228 is displayed. These screens provide a visual indication about the progress of the test. A typical test progress screen can include a certain shape that is gradually filled, according to the progress of the test. Various shapes can be selected. The inventors used the following shapes: heart, thermometer and upper part of a human body.

Once a test was completed a test result screen out of test result screens 230-240 is displayed. A test result screen indicates the test that was conducted and the result of the test. A test result screen can be followed (after a predefined time or after the user pressed a push control button) by a selective save screen 242 in which the user is asked whether he wants to store the result or not.

If the user selects (responsive to the main menu screen) to adjust the setting of compact monitor 10 then setting selection screen 250 is displayed. The user can select between setting the volume of buzzer 140 (“volume up/down”) or adjust the brightness of display 20 (“brightness up/down”). If the former is selected a volume adjustment screen 256 is displayed and the user can use the second and third push control buttons 54 and 56 to increase or decrease the volume of buzzer 140. If the latter is selected a brightness adjustment screen 258 is displayed and the user can use the second and third push control buttons 54 and 56 to increase or decrease the brightness of display 20.

If the user selects to review results of old tests (“history”), a result selection screen 260 is displayed and the user can select between viewing older results of various tests such as temperature test (“temp.”), ECG test (“ECG”), BFA test (“BFA”), heart rate test (“HR”) or exit to the main menu (“menu”). The selection is followed by a display of older test result screen out of screens 262-268.

Conveniently, a compact monitor is provided. The compact monitor includes a processor such as processor 112, a mass storage unit such as and a user interface such as control push buttons 52-56 and display 20. The compact monitor (such as compact monitor 10) is adapted to generate context-based reminders to carry out at least one predefined action, via the user interface, and to determine a completion of at least one predefined action in response to an input provided to the compact monitor. The input can be provided by the user. The input can be, for example, a text response that is inputted by using the control push buttons. The input can be a word selected from a group of predefined words. The input can also be an audio input that is sensed by a microphone (not shown) of compact monitor 10. The input can also involve contacting one or more physiological sensors and allowing the compact monitor to evaluate the state of the user and determine (in response to the evaluated state) whether the at least one predefined action was taken. The input can also be provided by elements other than the user. For example, if the user was requested to reach a certain place the input (indicating the location of the user) can be provided from location determining systems such as GRP systems.

In response to a completion of the at least one predefined action the compact monitor can take generate another reminder, and the like.

Compact monitor 10 can generate a context-based reminder in response to an environmental parameter, in response to a physiological or mental state of the user, in response to previously stored information (such as a definition of context and reminder that are at least partially provided by a host computer), and the like.

Various applications such as drug reminder and verifier applications can use the context-based features of compact monitor 10. A reminder to take a drug can be generated at predefined times. The user can be requested to confirm that the drug was taken and even can be encouraged to take a physiological test that can confirm that the drug was taken.

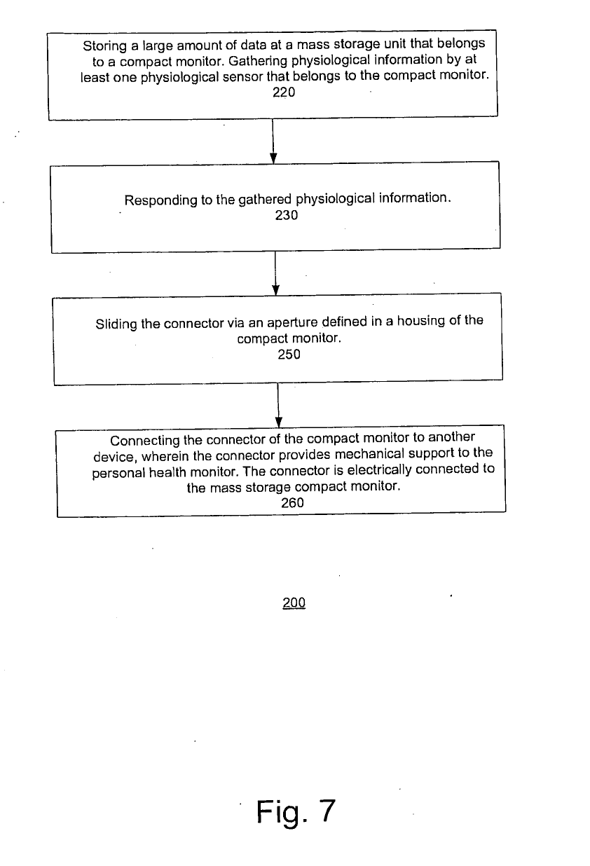

FIG. 7 illustrates method 300 for health monitoring, according to an embodiment of the invention.

Method 200 starts by stage 210 of storing a large amount of data (at least 8 mega-bytes) at a mass storage unit that belongs to a compact monitor. The data can include images, icons, text, results of old health tests, context information and the like. The mass storage unit can be a flash memory storage unit such as flash storage integrated circuit 132. The information can be provided in wireless manner (by a wireless transmitted included within a communication circuit), by using connector 70, and the like. The images and icons can be used during a monitoring application.

Stage 210 is followed by stage 220 of gathering physiological information by at least one physiological sensor that belongs to the compact monitor. Stage 220 can involve applying a first level of monitoring, applying a second level of monitoring, gathering physiological information without contacting the user, gathering physiological information while contacting the user, and the like.

Stage 220 is followed by stage 230 of responding to the gathered physiological information.

Stage 230 can include at least one of the following operations, or a combination thereof: (i) performing high-level processing by a high-level data processor that belongs to the personal health monitor; (ii) extracting physiological parameters in response to detection signals provided by at least one physiological sensor; (iii) outputting, by the high-level data processor, output information based upon the extracted physiological parameters; (iv) providing reminders or instructions responsive to at least one extracted parameter; (v) displaying information representative of the gathered physiological information on the display. It is noted that the displaying can include displaying the information on a graphic display.

Stages 220 and 230 can include conducting one or more tests, providing context based reminders (and verifying that the user acted according to the reminders), and the like. It is noted that multiple tests as well as multiple iterations of stages 220 and 230 can be executed before jumping to stage 250.

Stage 230 is followed by stage 250 of sliding the connector via an aperture defined in a housing of the compact monitor. The connector is positioned outside the housing in order to enable it to be connected to another device such as a host computer. Conveniently, stage 250 includes controlling the sliding by a control element (such as a slider) adapted to control a position of the connector.

Stage 250 is followed by stage 260 of connecting the connector of the compact monitor to another device, wherein the connector provides mechanical support to the personal health monitor; wherein the connector is electrically connected to the mass storage compact monitor.

Stage 260 usually involves reading information from the mass storage unit, downloading context information or old test results, downloading application code and the like.

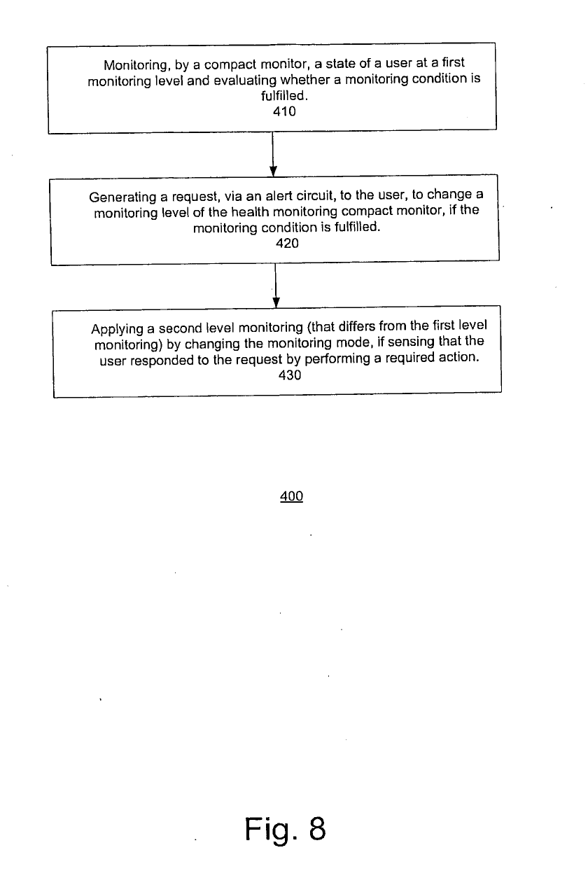

FIG. 8 illustrates method 400 for health monitoring, according to an embodiment of the invention.

Method 400 starts by stage 410 of monitoring, by a compact monitor, a state of a user at a first monitoring level and evaluating whether a monitoring condition is fulfilled.

If the monitoring condition if fulfilled stage 410 is followed by stage 420. The monitoring condition can represent at least one of the following: (i) a mental state of the user, (ii) an environmental condition, (iii) a heart beat of the user, (iv) a cardiac state of the user, (v) a respiration of the user.

It is noted that stage 410 can be conducted without connecting the user. For example, the compact monitor can conduct the first-level monitoring while being placed in a pocket of the user, within a handbag of the user, and the like. An exemplary contact-less monitoring can involve analyzing the voice of the user and determining whether the user is under stress.

Stage 420 includes generating a request, via an alert circuit, to the user, to change a monitoring level of the compact monitor, if the monitoring condition is fulfilled. The user can be requested to conduct a health test that requires the user to contact at least one physiological sensor of the compact device.

Stage 420 is followed by stage 430 of applying a second level monitoring (that differs from the first level monitoring) by changing the monitoring mode, if sensing that the user responded to the request by performing a required action. It is noted that the new monitoring level can be applied by utilizing the compact monitor but this is not necessarily so and another monitor can be used to apply the second level monitoring.

Conveniently, stage 430 can include testing the user's condition while the user contacts at least one physiological sensor of the compact monitor.

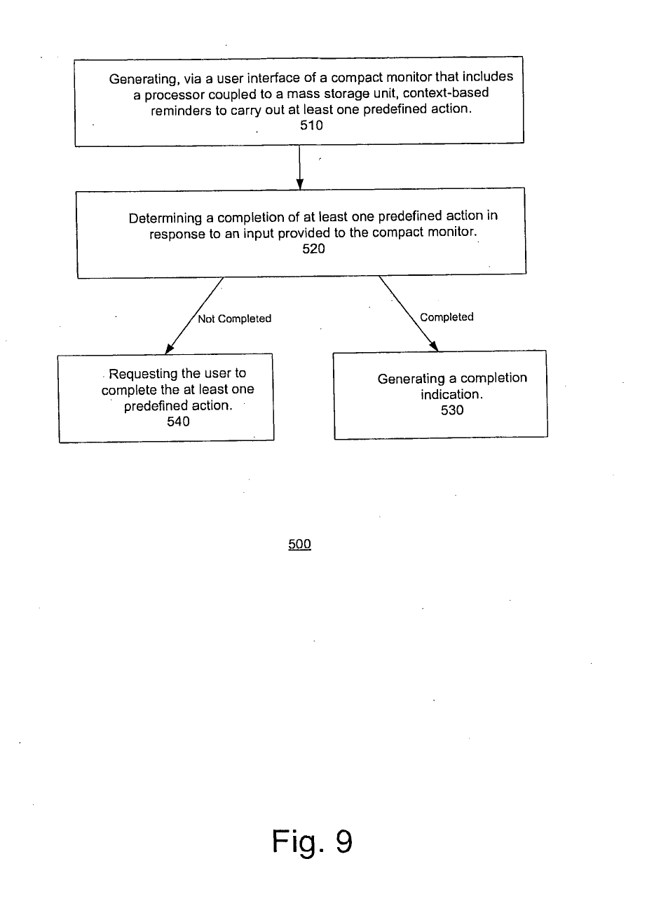

FIG. 9 illustrates method 500 for health monitoring, according to an embodiment of the invention.

Method 500 starts by 510 of generating, via a user interface of a compact monitor that includes a processor coupled to a mass storage unit, context-based reminders to carry out at least one predefined action.

Stage 510 is followed by stage 520 of determining a completion of at least one predefined action in response to an input provided to the compact monitor. The input can be provided by the user (using the control push buttons, contacting a sensor and the like), but this is not necessarily so. For example, location information can be provided by a location determining component that can be connected (even in a wireless manner) to the compact monitor, or can belong to the compact monitor.

If the at least one predefined action is completed stage 520 can be followed by stage 510 and another reminder can be generated. It is noted that if the at least one predefined action is completed stage 520 can be followed by stage 530 of generating a completion indication. Stage 510 can be followed by stage 530.

If the at least one predefined action is not completed then stage 520 can be followed by stage 540 of requesting the user to complete the at least one predefined action. Multiple iterations of 540 can be executed before method 500, and each iteration can involve displaying a different reminder to the user.

The context can be defined by at least one of the following: an environmental parameter, at least one signal provided by at least one physiological sensor, previously stored information, information provided by a host computer.

Various stages of method 500 can be executed by drug reminder and verifier application, diet monitoring application, fitness regime application and the like.

For example, a drug monitoring application may remind the user to take drugs and send him reminders until the appropriate drug is taken. The user can acknowledge taking the drug by using the control push buttons. Additional acknowledgment can involve testing the physiological state of the user and evaluating whether the drug was taken in response to the measured state of the user. If a fitness regime application is being executed the user can be requested to perform a certain physical action and the completion of that physical action can be verified by a location sensor, by heartbeat sensor and/or an accelerometer.

Those skilled in the art will readily appreciate that various modifications and changes may be applied to the preferred embodiments of the invention as hereinbefore exemplified without departing from its scope as defined in and by the appended claims.

We claim:

1. A compact monitor comprising:
at least one physiological sensor;
a mass storage unit; and
a connector, electrically coupled to the mass storage unit; wherein the connector is adapted to mechanically support the compact monitor when the connector is rigidly connected to a another device.

2. The compact monitor according to claim 1 further comprising a high-level data processor.

3. The compact monitor according to claim 2 wherein the high-level data processor is adapted to extract physiological parameters in response to detection signals provided by at least one physiological sensor.

4. The compact monitor according to claim 3 wherein the high-level data processor is adapted to output information based on the extracted physiological parameters.

5. The compact monitor according to claim 4 wherein the outputted information comprises outputting to a person reminders or instructions responsive to at least one extracted parameter.

6. The compact monitor according to claim 1 further comprising a display.

7. The compact monitor according to claim 6 wherein the display is a graphic display.

8. The compact monitor according to claim 1 wherein the compact monitor comprises a housing that includes an aperture through which the connector can slide.

9. The compact monitor according to claim 8 further comprising a control element adapted to control a position of the connector.

10. A method for health monitoring, the method comprises:
storing a large amount of data at a mass storage unit that belongs to a compact monitor;
gathering physiological information by at least one physiological sensor that belongs to the compact monitor; and
rigidly connecting a connector of the compact monitor to another device; wherein the connector provides mechanical support to the personal health monitor; wherein the connector is electrically coupled to the mass storage device.

11. The method according to claim 10 further comprising performing high-level processing by a high-level data processor that belongs to the personal health monitor.

12. The method according to claim 10 wherein the high-level processing comprises extracting physiological parameters in response to detection signals provided by the at least one physiological sensor.

13. The method according to claim 12 further comprising outputting, by the high-level data processor, output information based upon the extracted physiological parameters.

14. The method according to claim 13 further comprising providing reminders or instructions responsive to at least one extracted parameter.

15. The method according to claim 10 further comprising displaying information representative of the gathered physiological information on the display.

16. The method according to claim 15 wherein the displaying comprises displaying the information on a graphic display.

17. The method according to claim 10 further comprising sliding the connector via an aperture defined in a housing of the compact monitor.

18. The method according to claim 17 further comprising controlling the sliding by a control element adapted to control a position of the connector.

19. A compact monitor, comprising a processor coupled to an alert circuit and at least one physiological sensor;
wherein the compact monitor is adapted to monitor a state of a user at a first monitoring level and evaluate whether a monitoring condition is fulfilled; and
wherein in response to a completion of the monitoring condition the compact monitor is adapted to generate a request, via the alert circuit, to the user, to change a monitoring level of the compact monitor device.

20. The compact monitor according to claim 19 wherein the monitoring condition represents a mental state of the user.

21. The compact monitor according to claim 19 wherein the monitoring condition represents an environmental condition.

22. The compact monitor according to claim 19 wherein the at least one physiological sensor comprise a heart beat sensor.

23. The compact monitor according to claim 19 wherein the at least one physiological sensor comprise a cardiac state sensor.

24. The compact monitor according to claim 19 wherein the at least one physiological sensor comprise a respiration sensor.

25. The compact monitor according to claim 19 further adapted to change the monitoring mode, after the request is generated and the user performs a required action.

26. The compact monitor according to claim 19 wherein the compact monitor is adapted to apply the first monitoring level without contacting the user.

27. The compact monitor according to claim 26 wherein the compact monitor is adapted to apply a second monitoring level while the user contacts at least one physiological sensor.

28. The compact monitor according to claim 26 wherein the compact monitor is adapted to generate a request to the user to contact at least one physiological sensor.

29. A method for health monitoring, the method comprises:
monitoring, by a compact monitor, a state of a user, at a first monitoring level, and evaluating whether a monitoring condition is fulfilled; and
generating a request, via an alert circuit, to the user, to change a monitoring level of the compact monitor, if the monitoring condition is fulfilled.

30. The method according to claim 29 wherein the monitoring condition represents a mental state of the user.

31. The method according to claim 29 wherein the monitoring condition represents an environmental condition.

32. The method according to claim 29 wherein the monitoring comprises monitoring a heart beat of the user.

33. The method according to claim 29 wherein the monitoring comprises monitoring a cardiac state of the user.

34. The method according to claim 29 wherein the monitoring comprises monitoring a respiration of the user.

35. The method according to claim 29 further comprising changing the monitoring mode if sensing that the user responded to the request by performing a required action.

36. The method according to claim 29 wherein the monitoring comprises applying the first monitoring level without contacting the user.

37. The method according to claim 29 wherein the monitoring comprises applying a second monitoring level while the user contacts at least one physiological sensor.

38. The method according to claim 29 wherein the generating comprises generating a request to the user to contact at least one physiological sensor.

39. A compact monitor comprising:
a processor coupled to a mass storage unit and to a user interface;
wherein the compact monitor is adapted to generate context-based reminders to carry out at least one predefined action, via the user interface, and
wherein the compact monitor is adapted to determine a completion of at least one predefined action in response to an input provided to the compact monitor.

40. The compact monitor according to claim 39 wherein the compact monitor is adapted to generate a context-based reminder in response to an environmental parameter.

41. The compact monitor according to claim 39 further comprising at least one physiological sensor.

42. The compact monitor according to claim 41 wherein the processor is adapted to generate a context-based reminder in response to at least one signal provided by the at least one physiological sensor.

43. The compact monitor according to claim 42 wherein the processor is adapted to generate a context-based reminder in response to previously stored information.

44. The compact monitor according to claim 43 wherein the context is at least partially defined by information received from a computer.

45. The compact monitor according to claim 39 wherein the input is provided by the user and wherein the input is selected from a group of possible input words.

46. The compact monitor according to claim 39 further adapted to generate another reminder to the user if the at least one predefined condition was not fulfilled.

47. A method for monitoring a user, the method comprising:
generating, via a user interface of a compact monitor that comprises a processor coupled to a mass storage unit, context-based reminders to carry out at least one predefined action; and
determining a completion of at least one predefined action in response to an input provided to the compact monitor.

48. The method according to claim 47 wherein the generating is responsive to an environmental parameter.

49. The method according to claim 47 wherein the generating is responsive to at least one signal provided by at least one physiological sensor.

50. The method according to claim 47 wherein the generating is responsive to previously stored information.

51. The method according to claim 47 further comprising receiving information from a computer and wherein the generating is responsive to information received from the computer.

52. The method according to claim 47 wherein the receiving comprises receiving an input word that is selected from a group of possible input words.

53. The method according to claim 47 further comprising generating another reminder to the user if the at least one predefined condition was not fulfilled.

54. A compact monitor comprising:
at least one physiological sensor;
a mass storage unit; and
a communication circuit adapted to transmit physiological information gathered by the at least one physiological sensor.

55. The compact monitor according to claim 54 wherein the communication circuit is adapted to wirelessly transmit the physiological information.

56. A method for health monitoring, the method comprises:
storing a large amount of data at a mass storage unit that belongs to a compact monitor;
gathering physiological information by at least one physiological sensor that belongs to the compact monitor; and
transmitting the physiological information.

57. The method according to claim 56 wherein the transmitting comprises wirelessly transmitting the physiological information.
